(19) United States
(12) Patent Application Publication
(10) Pub. No.: US 2008/0177240 A1
(43) Pub. Date: Jul. 24, 2008

(54) Pyrogen-Free Neurosurgical Sponges

(75) Inventors: John E. Kemnitzer II, West Windsor, NJ (US); Karen Tuley, Cincinnati, OH (US); Simon J. Archibald, Pennington, NJ (US); Donna Wallace, Manchester, NJ (US); Peter Silvis, Flemington, NJ (US)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(73) Assignee: INTEGRA LIFESCIENCES CORPORATION, Plainsboro, NJ (US)

(21) Appl. No.: 11/914,499

(22) PCT Filed: May 16, 2006
(86) PCT No.: PCT/US06/18759

§ 371 Date: Nov. 15, 2007

Related U.S. Application Data
(60) Provisional application No. 60/681,260, filed on May 16, 2005.

(51) Int. Cl. A61F 13/36; B65D 71/00; B29C 67/20

(52) U.S. Cl. 604/368; 206/570; 264/321

(57) ABSTRACT

An improved neurosurgical sponge contains less than 0.45 EU/cm3 of bacterial endotoxins. A neurosurgical kit includes a plurality of sponges in a container, wherein the plurality of sponges contains less than 2.15 EU. Methods of producing the surgical sponge and using the surgical sponge in a surgical method are described. Rayon containing less than 0.45 EU of bacterial endotoxins per cm3 of rayon is also described.

### BACKGROUND OF THE INVENTION

1. Field of Invention

This invention relates to compositions for neurosurgery, and more particularly to low pyrogen content compositions suitable for use as neurosurgical sponges, and methods for making them.

2. Description of Related Art

Surgical sponges (a.k.a., neuropatties, surgical patties, neuropaddies, etc.) are used for a variety of purposes during surgical procedures. For example, surgeons employ such sponges to absorb blood and other body fluids and to isolate organs from the operating field. Surgical sponges employed in neurosurgery are known as neurosurgical sponges.

Despite the fact that neurosurgical sponges have been in use for many years, strict product specifications specifically for neurosurgical sponges have not been established by industry or government. The United States Food & Drug Administration (FDA) has, however, issued a guidance relating to medical devices in general, which is applicable to neurosurgical sponges. The 1987 publication of the FDA, entitled “GUIDELINE ON VALIDATION OF THE LIMULUS AMEBOCYTE LYSATE TEST AS AN END-PRODUCT ENDOTOXIN TEST FOR HUMAN AND ANIMAL PARENTERAL DRUGS, BIOLOGICAL PRODUCTS, AND MEDICAL DEVICES,” sets forth at page 11 the requirement that “[m]edical devices that contact cerebrospinal fluid should have less than 0.06 EU/ml of endotoxin.” Medical devices that meet this standard have been deemed “pyrogen-free”. Surgical sponges currently on the market have attempted to adhere to this standard.

However, the inventors have surprisingly discovered that the standard promulgated by the FDA is inadequate for ensuring the safety of neurosurgical sponges.

Accordingly, it is desired to provide improved neurosurgical sponges that address the deficiencies of the prior art.

All references cited herein are incorporated herein by reference in their entireties.

### BRIEF SUMMARY OF THE INVENTION

Accordingly, a first aspect of the invention comprises a surgical sponge comprising bacterial endotoxins in an amount less than 0.45 EU/cm³ of the sponge. The surgical sponge preferably comprises at least 50 wt. % rayon.

A second aspect of the invention comprises a neurosurgical kit, comprising a container and a plurality of surgical sponges within the container, wherein the plurality of sponges collectively contains bacterial endotoxins in a collective amount less than 2.15 EU.

A third aspect of the invention comprises a method of producing the surgical sponge of the invention, said method comprising the steps of:

(a) providing an absorbent material, wherein said absorbent material optionally contains bacterial endotoxins in a concentration of at least 0.45 EU/cm³ of absorbent material; and

(b) forming the absorbent material into a sponge,

provided that:

(1) when the concentration of bacterial endotoxins in the absorbent material is at least 0.45 EU/cm³ of absorbent material, pyrogens are extracted from the absorbent material into a solvent before and/or after the forming step; and

(2) when the concentration of bacterial endotoxins in the absorbent material is less than 0.45 EU/cm³ of absorbent material, the forming step is performed under pyrogen-free conditions and/or is followed by a pyrogen extraction step.

A fourth aspect of the invention comprises a method of using the sponge of the invention in a surgical procedure, said method comprising contacting bodily tissues with a plurality of the sponges to absorb bodily fluids from the bodily tissues, wherein the bodily fluids include cerebral spinal fluid and the plurality of sponges collectively contains less than 2.15 EU.

A fifth aspect of the invention comprises rayon comprising less than 0.45 EU of bacterial endotoxins per cm³ of rayon.

### DETAILED DESCRIPTION OF PREFERRED EMBODIMENTS OF THE INVENTION

The invention is based on our surprising discovery that prior art neurosurgical sponges may be both pyrogenic and cytotoxic as typically used by neurosurgeons. Individually, such sponges may be classified as “pyrogen-free” (in accordance with the above-described FDA definition for medical devices showing less than 0.060 EU/ml). However, neurosurgical sponges are sold in packages of multiple units, typically ten per package. A typical neurosurgical procedure comprises the use of as many as 100 sponges (more or less, depending upon the individual procedure), and the calculated EU load and cytotoxicity level anticipated would clearly exceed the FDA limits for this use, and may put the patient at risk for many complications of currently unknown origin.

Our study of currently marketed devices revealed that such devices may, either in single device units or multiple units (as typically employed), exhibit the undesirable characteristics of both pyrogenicity and cytotoxicity. The results of our study are summarized in Table I, and raise serious questions about possible patient safety issues.

TABLE I
Comparison Study of EO sterilized Neutec ½ ″× 6″ Surgical strips and
codman ½ ″× 6″ Surgical Strips.

| | | | REQUIREMENTS/ |
|---|---|---|---|
| TEST/ASSAY | NEUTEC | CODMAN | SPECIFICATIONS |
| Cytotoxicity | Severe cell | Moderate cell | Mild |
| | lysis/toxicity: | lysis/toxicity: | Grade 2 or less |
| | 100% lysis | 70% lysis | NMT 50% lysis |
| | Grade 4 | Grade 3 | |
| Particulates | | | None known |
| ≧10 μm | 55 | 58 | |
| ≧25 μm | 10 | 7 | |
| LAL | 0.080 EU/ml | 0.015 EU/ml | <0.060 EU/ml (FDA) |
| | 3.2 EU/device | 0.6 EU/device | ≦2.15 EU/device |
| | | (0.49 EU/cm³) | (USP) |
| DEHP | 53,000 ppm | 36,000 ppm | None known; |
| | | | Prop 65 NSRL = 80 μg/ |
| | | | day |
| Other Plasticizers | 150 ppm (DEHA) | None detected | None known |
| Darcy Permeability | 15.0 | 6.3 | No requirement |
| Constant | | | |
| Mean Flow Pore | 0.170 psi | 0.167 psi | No requirement |
| Pressure | | | |
| Mean Flow Pore Dia | 38 μm | 39 μm | No requirement |
| Largest Detected Pore | 92 μm | 89 μm | No requirement |
| Size | | | |
| Standard Deviation of | 23 μm | 24 μm | No requirement |
| Average Pore Dia | | | |
| Dia. @ Max Pore Size | 19 μm | 23 μm | No requirement |
| Dist | | | |
| Max Pore Size Dist | 2.1 | 2.1 | No requirement |
| Dia @ 10% | 78 μm | 74 μm | No requirement |
| Cumulative Filter | | | |
| Flow | | | |
| Dia @ 25% | 56 μm | 59 μm | No requirement |
| Cumulative Filter | | | |
| Flow | | | |
| Dia @ 75% | 23 μm | 25 μm | No requirement |
| Cumulative Filter | | | |
| Flow | | | |
| FTIR Comparison | Consistent with Rayon | Consistent with Rayon | No requirement |
| | (bulk match w/ | (bulk match w/ | |
| | Codman) | Neutec) | |
| As Received Pad | 9.0% | 8.5% | No requirement |
| Moisture (LOD) | | | |

Abbreviations used in Table I (and hereinafter):
USP, United States Pharmacopeia;
ppm, parts per million;
NMT, not more than;
μm, micrometer;
μg, microgram;
EU, endotoxin units;
ml, milliliter;
LAL, limulus amebocyte lysate;
psi, pounds per square inch;
NSRL, no significant risk level;
EO, ethylene oxide;
DEHP, di(ethyhexyl)phthlate;
DEHA, di(ethylhexyl)adipate;
LOD, loss on drying;
FDA, United States Food & Drug Administration;
dia, diameter; and Prop 65, California Proposition 65 - “The Safe Drinking Water and Toxic Enforcement Act of 1986”.

Abbreviations used in Table I (and hereinafter): USP, United States Pharmacopeia; ppm, parts per million; NMT, not more than; μm, micrometer; μg, microgram; EU, endotoxin units; ml, milliliter; LAL, limulus amebocyte lysate; psi, pounds per square inch; NSRL, no significant risk level; EO, ethylene oxide; DEHP, di(ethylhexyl)phthlate; DEHA, di(ethylhexyl)adipate; LOD, loss on drying; FDA, United States Food & Drug Administration; dia, diameter; and Prop 65, California Proposition 65—“The Safe Drinking Water and Toxic Enforcement Act of 1986”.

The study of Table I was conducted using a Kinetic-Chromogenic Limulus Amebocyte Lysate (KCLAL) Test in accordance with the following protocol. As per the FDA 1987 Guideline and USP convention for extraction volumes, each test unit was extracted with 40 ml of sterile nonpyrogenic water for injection. See USP Bacterial Endotoxins Test (USP 28/NF 23, 2005).

Each test unit was a 0.5 inch width×6 inch length surgical strip ranging in thickness between 0.02-0.03 inches. Seven units were covered with 280 ml of sterile nonpyrogenic water for injection and were extracted for 40-60 minutes at 37-40° C. in a shaker incubator. 0.1 ml of the test and control solutions was placed in a sterile microplate well and incubated at 37° C. for 10 minutes in a Kinetic-Chromogenic Reader.

0.1 ml of lysate, reconstituted per manufacturer's current directions, was then added to each well and testing commenced. The quantity of endotoxin, determined spectrophotometrically, and its reaction time, with the LAL/Substrate, was compared to that of a standard curve to determine endotoxin concentration.

A positive product control (PPC—inhibition/enhancement control) was simultaneously prepared to show that the test article did not significantly interfere with the lysate/substrate reaction. All procedures were conducted in conformance with good laboratory practice and ISO 17025.

Using these results on the Codman sponges, which pass the requirements for CSF contacting devices for a single unit, serves as a starting point for real-life calculations of endotoxin unit (EU) load. Assuming that 75 Codman sponges are soaked in 1 L of sterile saline solution prior to use, that each sponge absorbs 7 ml of saline solution (as determined experimentally), and that there is homogenization of EU distribution on soaking, we would expect that the patient will be exposed to 0.32 EU ((0.6 EU/device×75 devices×7 ml)/1000 ml) during the procedure. The value of 0.32 EU is only approximately 15% of the ≦2.15 EU/device maximum specified by the USP.

However, the foregoing calculation likely overemphasizes the effect of dilution in saline, because the sponges are simply placed in the solution and not really extracted with some sort of agitation. Reliance on such an undocumented effect puts the patient under life threatening risk. If we assume that the 75 sponges are used as removed from their original packaging, we would expect that the patient would be exposed to 45 EU (0.6 EU/device×75 devices) during the procedure. The value of 45 EU is 21 times greater than the ≦2.15 EU/device maximum specified by the USP.

Accordingly, one aspect of the invention comprises a neurosurgical sponge having reduced pyrogenicity. The term “neurosurgical sponge” as used herein is intended to encompass absorbent devices intended to contact cerebrospinal fluid during neurosurgical procedures. Shapes and sizes of the sponges are limited only by their intended application.

Materials from which sponges of the invention are formed are not particularly limited, provided that such materials are, or can be made, absorbent, substantially free of pyrogens and physiologically compatible. Suitable materials include, but are not limited to, cellulose, polyurethane, polyesters, polyester-cellulose blends, polypropylene-cellulose composites, poly(vinyl alcohol), and related copolymers and gels, cotton, poly(HEMA), crosslinked gels, collagen and the like. Most preferably, sponges of the invention predominantly comprise (i.e., contain more than 50 wt. %) rayon. In certain embodiments, the sponges comprise at least 5 wt. % collagen, or at least 10 wt. % collagen, or at least 25 wt. % collagen, or at least 50 wt. % collagen. In such embodiments, the collagen can comprise up to 100 wt. % collagen, or up to 90 wt. % collagen, or up to 80 wt. % collagen, or up to 75 wt. % collagen.

A material (or a device, such as a sponge, made from the material) is deemed to be “substantially free of pyrogens” for purposes of the present invention if the material contains bacterial endotoxins in an amount less than 0.45 EU/cm³ (note that the volume in cubic centimeters refers to the material tested, not the volume of extractant) as measured by the KCLAL test described above with respect to testing the Codman and Neutec products. Differences in the volume of the material tested should be taken into consideration during the extraction step. Approximately 40 ml of sterile nonpyrogenic water should be used for extraction per 1.23 cm³ of material being tested (i.e., about 32.5 ml water/cm³ of material). The amount of material tested is dictated by experimental considerations, and is preferably at least about 0.5 cm³.

Certain embodiments of the invention contain pyrogens in an amount less than 0.06 EU/ml (when extracted under specified conditions as per FDA guidance) and ≦2.15 EU/device (USP specification). When a plurality of such devices are used in a single procedure, then the total EU load must be taken into consideration, whereby the plurality of like devices used must be considered as a single unit, and not exceed that specified under FDA and USP requirements. This may be determined in one of several ways, such as by total number of such devices. This may be problematic because such devices come in a variety of sizes and shapes. A more preferable way is to determine endotoxin units per unit volume of material (EU/cm³ device material), as measured by the method described above with respect to evaluating the Codman and Neutec sponges. In this way, standardization of EU exposure potential for a given surgical procedure is known when using a plurality of sponges. Currently, this is not the case for surgical sponges, and more specifically neurosurgical sponges.

Thus, neurosurgical sponges of the invention are pyrogen-free for a specified number of units conservatively anticipated for use in neurosurgical procedures. In certain embodiments, a plurality of sponges packaged together collectively meets the FDA and/or the USP standards for a single medical device that contacts cerebrospinal fluid. Preferred embodiments of the sponges of the invention contain less than 0.45 EU/cm³, more preferably less than 0.25 EU/cm³, still more preferably less than 0.1 EU/cm³, and most preferably less than 0.025 EU/cm³ of device. All of the foregoing limitations are maximums. The minimum amount of bacterial endotoxins is not particularly limited. In certain embodiments, the minimum amount of bacterial endotoxins is 0.01 EU/cm³.

In addition to neurosurgical sponges having reduced pyrogenicity (such that they may be deemed “non-pyrogenic”), the invention also encompasses neurosurgical sponges having reduced cytotoxicity.

Additional testing has revealed that two common plasticizers, di(2-ethylhexyl)phthalate (DEHP, also known as di-octyl phthalate or DOP) and di(2-ethylhexyl)adipate (DEHA), and other currently unknown extractables are present in these commercially available products. DEHP has been classified as a possible male reproductive and carcinogenic risk. It is not unreasonable to suspect that the cytotoxicity results obtained may be related to DEHP, DEHA and/or other unknown extractables in the sponges tested. Certain embodiments of the invention exclude from the sponges plasticizing agents such as DEHP and/or DEHA.

In another aspect, the invention comprises a method of making neurosurgical sponges that are substantially free of pyrogens. Preferably, the method is simply an improvement over prior art methods for making surgical sponges, wherein the method is conducted under conditions and with materials that are substantially free of pyrogens. More preferably, the method is an improvement over prior art methods for making surgical sponges, wherein pyrogens are removed from the sponge and/or from the material from which the sponge is formed. The most preferred means for removing pyrogens is solvent extraction, such as liquid, gas or supercritical solvent extraction. The invention also encompasses a combination of the pyrogen-free and the pyrogen removal methods described above.

Methods and techniques for pyrogen reduction, removal and/or destruction are numerous and generally encompass at least one of the following: single or multiple filtration, dialysis, adsorption, anion exchange, dry heat, moist heat (autoclave), ionizing radiation (gamma, electron beam, X-ray), ethylene oxide and other sterilization methods, washing with/without pH control, digestion, masking by reaction, reverse osmosis, distillation, chromatography, and the like. Additional guidance regarding means for pyrogen reduction, removal and/or destruction is provided in U.S. Pat. No. 4,148,664; U.S. Pat. No. 4,588,400; U.S. Pat. No. 4,788,146; U.S. Pat. No. 4,808,314; U.S. Pat. No. 5,401,499; U.S. Pat. No. 5,948,409; U.S. Pat. No. 6,245,537; U.S. Pat. No. 6,599,518; U.S. Pat. No. 6,623,749; U.S. Pat. No. 6,774,102; U.S. Pat. No. 6,849,185; U.S. Pat. No. 6,858,179; and D. W. Cooper, “Reducing Pyrogens in Cleanroom Wiping Materials”, Pharmaceutical Engineering, Vol. 16, No. 4, 1996.

Another aspect of the invention comprises the production of rayon that is substantially free of pyrogens. This aspect of the invention is particularly useful in preparing sponges of the invention, but is by no means limited thereto. Rayon produced in accordance with the invention can be used for any application appropriate for conventional rayon (which is produced by processes that result in an abundance of pyrogens), but is particularly suitable for use in medical applications requiring a substantial lack of pyrogens. See, e.g., U.S. Pat. No. 6,599,518 and U.S. Published Patent Applications Nos. 2005/0042263; 2005/0042250; 2005/0019380; 2004/0161453; 2004/0142019; 2004/0028722; 2003/0203013; and 2003/0203012, which disclose the preparation and certain medical applications of cellulose materials having reduced pyrogen content.

In another aspect, the invention encompasses a surgical method, comprising the use of neurosurgical sponges of the invention. We believe that the invention will have an immediate and profound impact on reducing the current rates of various neurosurgical procedure complications, particularly where the cause of the complications have remained elusive to the neurosurgeon.

For example, aseptic meningitis is an extremely serious and common complication of neurosurgery. We believe that the use of prior art neurosurgical sponges is associated with the development of this condition as a complication of neurosurgery. For example, it has been reported only on posterior fossa surgery in children, the incidence of aseptic meningitis was 30% following surgery for Chiari malformation, 44% following operations for structural lesions and 25% following tumor surgery (Carmel et al., “The aseptic meningitis syndrome: a complication of posterior fossa surgery.” Pediatric Neurosurgery, 1993 September-October: 19(5):276-80). Additionally, the rate of deep post-operative sepsis, defined as ventriculitis, meningitis, brain abscess osteitis and subglial pus, was 4.1%, via a prospective study of 2249 neurosurgical cases (Narotam et al., “Operative Sepsis in Neurosurgery: A Method of Classifying Surgical Cases.” Neurosurgery, Vol. 34, No. 3 Mar. 1994). We have been unable to source any literature regarding the use of sponges for such procedures. We have also been unable to find what EU load will trigger a biological response (Ichinohe et al., “Usefulness of endotoxin-specific limulus test for the measurement of endotoxin in cerebrospinal fluid in diagnosis of bacterial meningitis.” Kansenshogaku Zasshi, 1995 November; 69(11):1227-34), probably because such responses may be dependent on many factors (dilution, route of administration, age and condition of patient). However, it has been documented that an intrathecal EU load may be much more toxic than intraveneously introduced. See Cooper et al., “Endotoxin as a cause of aseptic meningitis after radionuclide cisternography.” Journal of Nuclear Medicine, Vol 16, Issue 9 809-813, and Cooper, “Acceptance of the Limulus test as an alternative pyrogen test for radiopharmaceutical and intrathecal drugs.” Prog Clin Biol Res. 1979; 29:345-52. A cue may be taken from the pharmaceutical industry, which has strict guideline limits for EU content of bulk drugs.

The surgical method of the invention comprises contacting bodily tissues (e.g., brain, spinal cord, dura mater, arachnoid, pia mater, etc.) with a plurality of the sponges to absorb bodily fluids from the bodily tissues, wherein the bodily fluids include cerebral spinal fluid and the plurality of sponges collectively contains less than 2.15 EU. The number of sponges that can be used in the method is limited only by the limitation on the maximum exposure of the patient to endotoxins. The surgeon will typically use any number of sponges, e.g., 1 or 2 or 3 or 4 or 5 or 10 or 25 or 50 or more.

The kit of the invention is particularly well suited for use in conjunction with the surgical method of the invention. In its simplest form, the kit is simply a container containing at least one sponge of the invention. The container can be any material used for sterile packaging of medical devices. The kit can contain any number of sponges provided that the collective amount of endotoxins does not exceed a predetermined maximum amount. Collectively, the sponges of each kit preferably contain bacterial endotoxins in a collective amount less than 2.15 EU, more preferably less than 1.5 EU, and still more preferably less than 1 EU. The kit can contain, e.g., 1 or 2 or 3 or 4 or 5 or 10 or 25 or 50 or more sponges of the same or different sizes and shapes.

### Example 1

### Preparation of Rayon Fabric

Rayon fabric, prepared as typical by the industry, is subsequently subjected to extraction steps that facilitate the removal of contaminants, including pyrogens and potentially cytotoxic agents. These steps may include the use of pyrogen-free water (such as water for injection), 0.9% Saline (such as USP pyrogen free and sterile) or other pyrogen-free solvent systems that aid in the physical removal of pyrogens from the fabric. Additionally, subsequent processing steps that may be employed to give the fabric certain physical and handling characteristics must also make use of non-cytotoxic and pyrogen-free additives, including any solvent treatments (water and the like). Cutting, finishing, packaging and sterilization operations must maintain the level of cleanliness dictated by the FDA for the manufacture of medical devices, and more specifically for medical devices manufactured for use in neurosurgery. At any step along the way, fabric may be removed for endotoxin evaluation.

### Example 2

### Preparation of Sponges

Neurosurgical sponges, such as those composed of rayon, may be subjected to extraction steps to facilitate the removal of contaminants, including pyrogens and potentially cytotoxic agents. The method of the USP Bacterial Endotoxins Test, employing sterile nonpyrogenic water for injection, is considered an efficient extraction medium and method, such that the endotoxins are efficiently transferred into the extraction medium. The efficiency of removal can then be determined by measuring the endotoxin content of the successive extractions until a satisfactory level is achieved. The sponges may then be dried and further processed as dictated by manufacturing protocols (such as packaging and sterilization).

While the invention has been described in detail and with reference to specific examples thereof, it will be apparent to one skilled in the art that various changes and modifications can be made therein without departing from the spirit and scope thereof.

What is claimed is:

1. A surgical sponge comprising bacterial endotoxins in an amount less than 0.45 EU/cm³ of the sponge.

2. The surgical sponge of claim 1, wherein the sponge comprises at least 50 wt. % rayon.

3. The surgical sponge of claim 1, wherein the sponge comprises at least 50 wt. % collagen.

4. The surgical sponge of claim 1, wherein the amount of bacterial endotoxins is less than 0.25 EU/cm³ of the sponge.

5. The surgical sponge of claim 1, wherein the amount of bacterial endotoxins is less than 0.1 EU/cm³ of the sponge.

6. A neurosurgical kit, comprising a container and a plurality of sponges of claim 1 within the container, wherein the plurality of sponges collectively contains bacterial endotoxins in a collective amount less than 2.15 EU.

7. The kit of claim 6, wherein each of the sponges comprises at least 50 wt. % collagen.

8. The kit of claim 6, wherein each of the sponges contains bacterial endotoxins in an amount less than 0.25 EU/cm³ of the sponge.

9. The kit of claim 6, wherein each of the sponges contains bacterial endotoxins in an amount less than 0.1 EU/cm³ of the sponge.

10. The kit of claim 6, wherein there are at least 5 sponges.

11. The kit of claim 6, wherein there are at least 10 sponges.

12. The kit of claim 6, wherein the collective amount of bacterial endotoxins is less than 1.5 EU.

13. A method of producing the surgical sponge of claim 1, said method comprising the steps of:
(a) providing an absorbent material, wherein said absorbent material optionally contains bacterial endotoxins in a concentration of at least 0.45 EU/cm³ of absorbent material; and
(b) forming the absorbent material into a sponge,
provided that:
(1) when the concentration of bacterial endotoxins in the absorbent material is at least 0.45 EU/cm³ of absorbent material, pyrogens are extracted from the absorbent material into a solvent before and/or after the forming step; and
(2) when the concentration of bacterial endotoxins in the absorbent material is less than 0.45 EU/cm³ of absorbent material, the forming step is performed under pyrogen-free conditions and/or is followed by a pyrogen extraction step.

14. The method of claim 13, wherein the concentration of bacterial endotoxins is at least 0.45 EU/cm³, and the pyrogens are extracted.

15. The method of claim 13, wherein the concentration of bacterial endotoxins is less than 0.45 EU/cm³, and the forming step is performed under pyrogen-free conditions and/or is followed by a pyrogen extraction step.

16. The method of claim 13, wherein the sponge comprises at least 50 wt. % collagen.

17. The method of claim 13, wherein the amount of bacterial endotoxins is less than 0.25 EU/cm³ of the sponge.

18. The method of claim 13, wherein the amount of bacterial endotoxins is less than 0.1 EU/cm³ of the sponge.

19. A method of using the sponge of claim 1 in a surgical procedure, said method comprising contacting bodily tissues with a plurality of the sponges to absorb bodily fluids from the bodily tissues, wherein the bodily fluids include cerebral spinal fluid and the plurality of sponges collectively contains less than 2.15 EU.

20. Rayon comprising less than 0.45 EU of bacterial endotoxins per cm³ of rayon.

21. The rayon of claim 20, comprising less than 0.25 EU of bacterial endotoxins per cm³ of rayon.
